(19) United States
(12) Patent Application Publication
(10) Pub. No.: US 2014/0005793 A1
(43) Pub. Date: Jan. 2, 2014

(54) NOVEL BIOLOGICAL IMPLANT COMPOSITIONS, IMPLANTS AND METHODS

(71) Applicants: Keith Cameron Koford, Gainesville, FL (US); Nathaniel Scott Long, Newberry, FL (US); Erica Lee Scharringhausen, Gainesville, FL (US); Randall Keith Hilliard, Gainesville, FL (US)

(72) Inventors: Keith Cameron Koford, Gainesville, FL (US); Nathaniel Scott Long, Newberry, FL (US); Erica Lee Scharringhausen, Gainesville, FL (US); Randall Keith Hilliard, Gainesville, FL (US)

(21) Appl. No.: 13/795,976

(22) Filed: Mar. 12, 2013

Related U.S. Application Data
(60) Provisional application No. 61/662,749, filed on Jun. 21, 2012.

(51) Int. Cl. A61L 27/50; A61F 2/46; A61L 27/56

(52) U.S. Cl. CPC A61L 27/50; A61L 27/56; A61F 2/4601
USPC 623/23.5; 623/23.61; 623/23.63; 623/16.11; 264/344

(57) ABSTRACT

The present application is directed to the field of implants comprising processed hard or soft biological tissues for use in implantation in humans. The molded biological tissue implants of the present application are preferably made from allograft soft tissue sources and demineralized bone matrix. The present application provides biological implants exhibiting advantageous properties of absorption, expansion, resiliency and shape retention. The properties of the biological implants produced by the methods of the present application are leveraged in the creation and development of novel biological implant constructs and of novel methods of treatment and surgical technique.

### RELATED APPLICATIONS

This application relates to and claims priority benefits from U.S. Provisional Patent Application Ser. No. 61/662,749, filed Jun. 21, 2012, entitled “Novel Biological Implant Compositions, Implants and Methods”. The '749 provisional application is hereby incorporated by reference in its entirety.

### FEDERALLY SPONSORED RESEARCH OR DEVELOPMENT

[Not Applicable]

### FIELD OF THE INVENTION

The present application is directed to the field of biological tissue implants and biological tissue implant processing for transplantation, preferably into humans. The tissue implants are molded tissue implants (grafts), preferably made from allograft soft tissue sources and demineralized bone matrix. The present application provides biological implants exhibiting advantageous properties such as absorption, expansion, resiliency and shape retention. The properties of the biological implants produced by the methods of the present application are leveraged in the creation of novel biological implants and constructs and also in novel methods of treatment and surgical techniques.

### BACKGROUND OF THE INVENTION

Many injuries and ailments throughout the human body are treated through surgical intervention utilizing either biological tissue or synthetic material implants. Among these are conditions including spinal degeneration, sports medicine, podiatric, trauma and general orthopedic injuries or maladies involving bone or hard tissue. Also commonly benefiting from surgical intervention utilizing either biological tissue or synthetic material implants are soft tissue conditions including hernia, urological, gynecological, cardiac, neural, and general abdominal injuries or maladies.

In selecting implants to address these various injuries and maladies, surgeons are often faced with tradeoffs between natural, biological tissue materials and synthetic materials. Biological materials often offer natural healing, incorporation and regenerative capability, but may be lacking in material properties or handling characteristics as compared to synthetics. Surgeons must therefore often choose to accept the lack of regeneration and incorporation potential in using a synthetic material, which is at best inert and at worst inflammatory and prone to infection, in order to find an implant with the physical handling and material properties required for a particular surgical application. This is true in the growing area of minimally invasive surgical procedures, where synthetic material may offer strength, resiliency, compressibility, expansion, or an ability to fold or roll into a compact or compressed form for passage through a portal or delivery instrument before expanding, unfolding or unrolling at an implant site. Synthetic implants also offer a greater range of coating and combining materials due to their ability to bond and join together more efficiently than has been shown with natural materials.

The present application is directed at developing a biological material that exhibits advantageous properties such as fluid absorption, compressibility, expansion, resiliency and shape retention, while allowing for good regeneration and incorporation.

### BRIEF SUMMARY OF THE INVENTION

One embodiment of the present biological graft, consisting essentially of human tissue components is made by molding the graft into a first configuration and dehydrating the graft, rehydrating the graft with a first rehydration fluid and compressing the graft into a second configuration suitable for passage into a human patient wherein the second configuration is smaller in at least one dimension than the first configuration and dehydrating, packaging and sterilizing the graft in said second configuration. The graft can be rehydrated from the second configuration using a second rehydration fluid. The first and second rehydration fluids can be water, blood, blood components, saline, platelet rich plasma or bone marrow aspirate. The first and second rehydration fluids can be the same or different fluids. The graft can expand back to the first configuration upon application to a surgical site within the human patient. The first configuration can be, for example, a square, rectangle, circle, triangle or oval.

One embodiment of the present method of surgery on a human patient using the graft described above comprises providing the dehydrated, packaged and sterilized graft in the form of a compressed sheet having an initial dehydrated thickness and an initial dehydrated shape, and passing said graft into a surgical site within said human patient through a minimally invasive access portal having a cross sectional area, and rehydrating and expanding said graft at said surgical site within said human patient, and positioning said graft at the surgical site covering an implant area that is larger than said cross sectional area of said portal, and fully rehydrating said graft, thereby causing said graft to return to a hydrated thickness greater than that of said initial dehydrated thickness.

One embodiment of the present method of surgery on a human patient comprises providing an allograft unitary implant consisting essentially of human tissue components in a compressed dehydrated form, and providing a minimally invasive access portal into a surgical site within the human patient, and passing said implant through the minimally invasive access portal, and providing a hydration fluid in contact with the implant, wherein the implant expands following contact with the hydration fluid to create an expanded implant, and positioning or fixing the implant in place at the surgical site to create a fixed implant, wherein, the expanded implant has a fixed unitary shape which is larger than that which could have passed through said portal. The allograft unitary implant can be a molded tissue. The molded tissue can be demineralized bone matrix (DBM) or a combination of DBM and a component derived from a human tissue slurry. The allograft unitary implant can be rolled in a compressed dehydrated form. The expanded implant can be for example, a square, rectangle, circle, triangle or oval, as well as any regular or irregular shape suitable for or required by the specific surgical procedure or anatomical treatment site.

One embodiment of the present biological graft, consisting essentially of human tissue components comprises a bone block of cortical bone, cancellous bone or both, the bone block having at least one porous or semi-porous surface and a molded tissue component comprising demineralized bone matrix (DBM) and a human tissue slurry wherein the molded tissue component is integrated into the porous or semi-porous surface of the bone block. The molded tissue component can be frozen and lyophilized along at least a portion of at least one outer surface of the bone block. The bone block can also comprise at least one slot, groove or hole to increase penetration of the molded tissue component. The molded tissue component can cover at least two sides of the bone block and substantially fill a passageway between at least two of the at least two sides. The bone block can be in the shape of a wedge, trapezoid, plank or ring.

An embodiment of the present dehydrated, packaged and sterilized molded human tissue graft is in the form of a compressed sheet having an initial dehydrated thickness and an initial dehydrated compressed shape wherein the graft in the initial dehydrated compressed shape is small enough to be passed into a surgical site within a human patient through a minimally invasive access portal having a cross sectional area, and wherein the graft in said initial dehydrated compressed shape is rehydratable to return to a hydrated thickness greater than that of the initial dehydrated thickness at the surgical site, and to a hydrated size larger than the cross-sectional area.

In one aspect, the present application is directed to a molded biological all human tissue graft for use in a human patient. The molded tissue implant (graft) is derived from a processed natural biological tissue source, such as xenograft, allograft, or autograft tissue for human implantation, allograft is preferred. An implant of the present application may be made from a soft tissue source such as dermis, fascia or tendon, in combination with demineralized bone matrix (DBM). An implant of the present application may also contain additional hard tissue materials such as bone, bone particles or bone fibers which have or have not been demineralized by methods known in the art. Preferred implants consist essentially of human tissue components. Preferred materials are osteoconductive, compressible, conformable, and hydratable.

In one embodiment, the present application is directed to a processed minimally invasive biological graft for implantation into a human patient made from molded tissue components in a dehydrated state. In this embodiment the molded tissue graft is compressed into a first configuration suitable for passage into a human patient and is further capable of opening and expansion to a second configuration suitable for application to a surgical site within the human patient. In this embodiment the second configuration is larger in at least one dimension than the first configuration. As described in greater detail below, the compression is preferably achieved via rolling, folding, spiraling, winding or crumpling, for example. The compression occurs in one or more dimensions and the expansion is due, at least in part, to rehydration of the graft. This allows for implantation of larger grafts while still using minimally invasive procedures.

In another embodiment, the present application is directed to an assembled bone allograft implant comprising two or more bone blocks which are substantially planar segments of cortical bone, cancellous bone or both, which also contains at least one component that is a molded tissue component. The molded tissue component can comprise at least one layer of substantially non-bone human allograft tissue which is osteoconductive, compressible, conformable, and hydratable. The molded tissue component is formed from a tissue slurry of substantially of non-bone origin together with demineralized bone matrix (DBM). As described in greater detail below, the molded tissue component can be sandwiched between at least two substantially planar segments of cortical bone, cancellous bone or both. In these embodiments, the bone components can be in the shape of a plank. In some embodiments the assembled implant also has at least one bone pin holding the assembled graft together. The pin can be made from cortical or cancellous bone.

In another embodiment, the present application is directed to a unitary allograft bone block implant comprising a block of cortical bone, cancellous bone or both; and a molded tissue component which is integrated into the block. Optionally, the implant also has a compression zone formed by the molded tissue component, outside of the bone graft and adjacent to the machined outer surface. As described in greater detail below, in one embodiment, the bone block is a wedge or trapezoid and the molded tissue component is formed along at least one outer surface of the wedge or trapezoid. In other embodiments, the bone block is in the shape of a ring and the molded tissue component can fill all or some of the space inside.

The present application is additionally directed to methods of surgery on a human patient using a compressed and dehydrated graft (or unitary implant). In one embodiment, the grafts have the form of a compressed sheet having an initial dehydrated thickness and an initial dehydrated shape. Preferably, the graft is passed into a surgical site through a minimally invasive access portal. In these embodiments, the graft is expanded at the surgical site within the patient, and the implant covers an implant surgical site area that is larger than said cross sectional area of the minimally invasive access portal. Preferably, the expansion is due to fully or partially rehydrating the graft, thereby causing the graft to return to a hydrated thickness greater than that of the dehydrated thickness. Preferably, the minimally invasive access portal has a cross sectional area and the graft covers an implant area that is larger than the portal cross sectional area. More preferably, the graft or implant expands following contact with a hydration fluid and the expanded implant has a fixed unitary shape or configuration which is larger than that which could have passed through the portal. Preferred shapes or configurations are a square, rectangle, circle, triangle or oval. Optionally the implants (grafts) are packaged and sterilized.

These and other advantages and novel features of the present application, as well as details of illustrated embodiments thereof will be more fully understood from the following description of the drawings

### BRIEF DESCRIPTION OF THE SEVERAL VIEWS OF THE DRAWINGS

FIGS. 1A-1F show one embodiment of the present surgical method of a minimally invasive procedure using a rolled compressed dehydrated packaged sterilized molded tissue graft comprising demineralized bone together with a human tissue slurry. FIG. 1A shows a molded tissue graft having a rolled compressed configuration contained within a package. FIG. 1B shows a molded tissue graft being inserted through a minimally invasive portal into a minimally invasive surgical opening. FIG. 1C shows a molded tissue graft passing through a minimally invasive portal. FIG. 1D shows a molded tissue graft having passed through a minimally invasive portal into a patient body and lying adjacent a target surgical tissue being hydrated by a hydration fluid through a hydration needle being supplied by a hydration source. FIG. 1E shows a rolled compressed molded tissue graft unrolling to a partially open configuration following hydration inside a patient body and adjacent a target surgical tissue. FIG. 1F shows a rolled compressed molded tissue graft essentially completely unrolled to an open configuration following hydration and affixed to a target surgical tissue by fixation means.

FIG. 2 shows one embodiment of the present folded, compressed, dehydrated, packaged and sterilized molded tissue graft comprising demineralized bone together with a human tissue slurry.

FIGS. 3A-3C show one embodiment of the present molded tissue graft in three states: FIG. 3A—initial solid dehydrated state; FIG. 3B—rehydrated, compressed, rolled and finally dehydrated state; and FIG. 3C—secondarily rehydrated, expanded and unrolled state with fixation at a surgical site within a patient.

FIGS. 4A and 4B show one embodiment of the present composite bone block wedge of human bone together with a molded tissue component. A top view is shown in FIG. 4A and a section view through the line A-A is shown in FIG. 4B.

FIGS. 5A and 5B show one embodiment of the present composite bone block wedge of human bone together with a molded tissue component having discreet retention features along one surface. A top view is shown in FIG. 5A and a section view through the line A-A is shown in FIG. 5B.

FIGS. 6A and 6B show one embodiment of the present composite bone block wedge of human bone together with a molded tissue component having passageways in communication between two surfaces. A top view is shown in FIG. 6A and a section view through the line A-A is shown in FIG. 6B.

FIGS. 7A and 7B show one embodiment of the present composite bone implant having cortical bone, cancellous bone and a molded tissue components assembled to form an assembled implant. A section view through the line A-A is shown in FIG. 7A and top view is shown in FIG. 7B.

FIG. 8 shows one embodiment of the present composite bone implant having a molded tissue component molded between and completely surrounded by only two pieces of cortical bone.

FIG. 9 shows one embodiment of the present composite bone implant having a molded tissue component molded between and contacting at least 4 distinct pieces of cortical bone.

FIG. 10 shows one embodiment of the present composite bone implant having a molded tissue component molded between and contacting at least 6 distinct pieces of cortical bone.

FIG. 11 shows one embodiment of the present composite bone implant having a molded tissue component sandwiched between and contacting at least 2 cancellous bone planks.

FIG. 12 shows one embodiment of the present composite bone implant having a molded tissue component sandwiched between and contacting at least 1 cancellous bone plank and at least 1 cortical bone plank.

FIGS. 13A and 13B show one embodiment of the present composite bone implant having a molded tissue component molded into a machined recess within a bone plug. A top view is shown in FIG. 13A and a section view through the line A-A is shown in FIG. 13B.

FIG. 14 shows one embodiment of the present composite allograft cortical bone strut implant with a molded tissue component covering one side of the cortical bone strut.

The foregoing summary, as well as the following detailed description of certain embodiments of the present application, will be better understood when read in conjunction with the appended drawings. For the purposes of illustration, certain embodiments are shown in the drawings. It should be understood, however, that the claims are not limited to the arrangements and instrumentality shown in the attached drawings. Furthermore, the appearance shown in the drawings is one of many ornamental appearances that can be employed to achieve the stated functions of the system.

### DETAILED DESCRIPTION OF THE INVENTION

In one aspect, the present application is directed to the field of biological tissue implants and biological tissue implant processing for transplantation in a human or non-human animal species. In a preferred embodiment, the present application is directed to a molded biological tissue implant for use in a human patient. The implant is derived from a processed natural biological tissue source, such as xenograft, allograft, or autograft tissue for human implantation. Preferred molded grafts consist essentially of human tissue components. An implant of the present application may be processed from a soft tissue source such as dermis, fascia or tendon. Preferred materials are osteoconductive, compressible, conformable, and hydratable. These implants have the potential for enhanced mechanical properties. The terms “graft” and “implant” are used interchangeably herein.

As used herein, the term “passivate” is intended to refer to the elimination of potentially pathogenic organisms and immunogenic substances from an implant. Thus, both sterility and reduced antigenicity is intended by this term, although elimination of beneficial biological properties of the implant, such as osteogenic properties (osteoconduction or osteoinduction; bone fusion), natural tissue functionality, and desirable structural strength of an implant are not intended by this term. The term “passivation” is preferred to the term “sterilize” because, while sterilization is a goal, that term has an absolute connotation for which the ability to definitively test is limited by the state of the art of making such measurements and/or by the need for attendant tissue destruction. In addition, while the implants produced according to the method of this application may not be completely devoid of any antigenicity or pyrogenicity, these undesirable aspects are greatly reduced, and this too is intended by the term “passivation,” as used herein.

To be suitable for implantation in humans, the grafts (implants) of the present application must be treated to neutralize, remove or substantially reduce antigenic proteins, which may generate a rejection of the implant. It also must be treated to neutralize, remove or substantially reduce bacteria and viruses. Suitable processes for removing antigenic proteins and sterilizing to neutralize any bacteria and viruses are known in the art. See U.S. Pat. No. 5,846,484, entitled “Pressure flow system and method for treating a fluid permeable workpiece such as a bone,” which issued to Scarborough, et al. on Dec. 8, 1998. In the present case, the applicants utilized the assignees' method for defatting tissue, which also has the added benefit of removing blood, cellular debris, and soluble and antigenic proteins, by subjecting the muscle tissue to alternating cycles of pressure and vacuum in the sequential presence of solvents, such as isopropyl alcohol, hydrogen peroxide and a detergent. These assignee's processes also neutralize any bacteria and viruses. These processes are disclosed in full detail in assignee's U.S. Pat. No. 6,613,278, entitled “Tissue Pooling Process,” which issued to Mills et al., on Sep. 2, 2003; U.S. Pat. No. 6,482,584, entitled “Cyclic implant perfusion cleaning and passivation process,” which issued to Mills, et al. on Nov. 19, 2002; and U.S. Pat. No. 6,652,818, entitled “Implant Sterilization Apparatus,” which issued to Mills et al., on Nov. 25, 2003, all of which are incorporated herein by reference in their entirety.

“Soft tissue”, as used herein, refers to any biological tissue other than bone, including but not limited to tendons, ligaments, fascia, whole joints, dura, skin, pericardia, heart valves, veins, neural tissue, submucosal tissue (e.g. intestinal tissue), and cartilage, or a combination thereof. The “soft tissue” described herein is typically a collagenous material that is autograft, allograft or xenograft, preferably allograft. By definition, a “tendon” is a collagenous cord that attaches muscle to its point of origin, typically to bone. By definition, a “ligament” is a band of collagenous tissue that connects bone or supports viscera. However, such terms are used somewhat interchangeably in the implant art and by recitation of tendon, it is intended to encompass the use of ligament as well. Soft tissue may comprise a component of the present grafts and is preferably processed into a carrier.

“Graft” (or “implant”), as used herein, refers to any material the implantation of which into a human or an animal is considered to be beneficial. Accordingly, the implant may be tissue-derived material, such as bone, skin, and the like, or it may be a metallic or synthetic material, or any combinations thereof. An implant may comprise autograft tissue, allograft tissue, xenograft tissue or combinations thereof, and in the case of mineralized tissues, such as bone, the implant may comprise mineralized tissue, partially demineralized tissue, completely demineralized tissue, and combinations thereof. Preferred are grafts made substantially of only allograft tissue. Preferred embodiments include only human tissue. Such embodiments are preferred because minimally manipulated human tissue for homologous use may experience more favorable regulatory review.

The implant may comprise unitary or monolithic graft material, assembled bone materials such as those described in U.S. patent application Ser. Nos. 09/782,594 and 09/941,154, shaped implants such as those described in U.S. Pat. Nos. 6,440,444 and 6,696,073, and allogeneic biocompatible matrices such as those described in U.S. patent application Ser. Nos. 10/754,310 and 10/793,976. The present processes and apparatus may also be employed in the treatment of implants such as those described in U.S. Pat. Nos. D461,248; 6,290,718; 6,497,726; 6,652,592; 6,685,626; and 6,699,252. All of the foregoing patents and patent applications are incorporated by reference herein.

“Osteoconductive”, as used herein, refers to guiding the growth of natural bone; that is, the ability of a substance to serve as a template along which bone may grow. Osteoconductivity is typically associated with a natural pore size, surface characteristic and micro-topology such as that found in collagen constructs.

The present application provides specific steps to create molded biological tissue grafts (implants) for use in human patients. Implants are preferably made from allograft tissues and are osteoconductive, compressible, conformable, and hydratable. An implant of the present application may be processed from a soft tissue source such as dermis, fascia or tendon which is made into a tissue slurry and then combined with demineralized bone matrix (DBM). Implants can be molded at least one time, and possibly multiple times, and dehydrated, at least one time, and possibly multiple times. Grafts can be rehydrated for implantation or during processing, at least one time, and possibly multiple times.

Dehydration is part of the molding process of the grafts, and also can provide a step where a graft is held in a minimally invasive form. After dehydration, implants are rehydrated, at least one time, and possibly multiple times. Rehydration can occur as part of the processing of the grafts, and/or at the time of implantation (either before or after actual implantation into a patient). Rehydration fluids can be water, blood, blood components, saline, platelet rich plasma, bone marrow aspirate or combinations thereof. In one embodiment, two rehydrations are performed, and the second rehydration fluid and the first rehydration fluid are different or the same. Optionally the implants are dehydrated, packaged and sterilized, preferably in a minimally invasive configuration (after a second dehydration).

One example of a mold for the grafts of the application is about 25 mm wide by about 75 mm long by about 5 mm deep. Mold lengths of about 1-1000 mm, widths of about 1-500 mm and depths (height) of about 0.5-30 mm are contemplated. Disks of about 1-200 mm diameter and depths (height) of about 0.5-30 mm are also contemplated. In certain embodiments mold lengths of about 50-1000 mm, widths of about 5-500 mm and depths (height) of about 5-30 mm may be used. In certain embodiments disks of about 20-200 mm diameter and depths (height) of about 5-30 mm may be used. In certain embodiments mold lengths of about 30-100 mm, widths of about 10-30 mm and depths (height) of about 5-10 mm may be used. In certain embodiments disks of about 10-100 mm diameter and depths (height) of about 5-30 mm may be used. In certain embodiments mold lengths of about 5-50 mm, widths of about 1-50 mm and depths (height) of about 1-10 mm may be used. In certain embodiments disks of about 5-50 mm diameter and depths (height) of about 1-10 mm may be used. In certain embodiments mold lengths of about 2-15 mm, widths of about 2-10 mm and depths (height) of about 1-10 mm may be used. In certain embodiments disks of about 2-20 mm diameter and depths (height) of about 1-10 mm may be used. In certain embodiments mold lengths of about 1-10 mm, widths of about 1-10 mm and depths (height) of about 0.5-5 mm may be used. In certain embodiments disks of about 1-10 mm diameter and depths (height) of about 0.5-5 mm may be used. Molded grafts are dried (dehydrated), by lyophilization or other means known in the art. Molded grafts are optionally frozen prior to or as part of the lyophilization or drying cycle. Freezing time, drying conditions, temperature, airflow and physical conditions may be controlled to influence graft properties. For example, a conventional freezer may be used to bring molded grafts to a frozen state prior to lyophilization in minutes to a few hours depending on graft size, geometry, packaging and placement. Alternatively, a programmed cycle may be set to bring the grafts to freezing very quickly, in a matter of minutes, sometimes in 5-10 minutes, alternatively in 10-20 minutes, alternatively 20-30 minutes, alternatively 30-60 minutes. Molded grafts of certain configurations may be flash-frozen in less than 10 minutes, alternatively less than 5 minutes, alternatively less than 1 minute. Molded grafts may be slowly frozen with controlled, limited or accelerated air drying over a period of 1-2 hours, alternatively 2-4 hours, alternatively 4-8 hours or longer. Molded implants may be air dried or partially air dried prior to freezing. Molded implants may be molded in layers of the same or different compositions and under the same or different drying, freezing and lyophilizing conditions for each layer. The grafts are resistant to tearing during surgical implantation or pre-surgical preparation, but can be cut or torn under specifically focused manual pressure either at discrete locations coincident with features such as a one or more notches, perforations or gaps, or at one or more non-predetermined locations, for ease in surgery.

The tissue slurry that is used to make the grafts of the present application uses a liquid component. The liquid component is acidic, basic or neutral. Typically, the ratio of liquid component (volume) to dry weight of tissue (grams) is within the range of 100:1 to 5:1; alternatively within the range of 80:1 to 20:1; alternatively within the range of 70:1 to 30:1; alternatively within the range of 60:1 to 40:1; alternatively within the range of 40:1 to 5:1; alternatively within the range of 10:1 to 30:1; alternatively within the range of 25:1 to 15:1; alternatively within the range of 90:1 to 50:1; alternatively within the range of 80:1 to 60:1; alternatively within the range of 75:1 to 65:1. The choice of the ratio of liquid component to tissue determines the viscosity of the slurry and the choice is based upon the ultimate application of the slurry. To make the slurry, tissue is comminuted (macerated, shredded, chopped, etc.) and mixed with the liquid component. Mixing continues until the slurry has uniform consistency. Slurries may be degassed. Degassing can be accomplished by methods known in the art, including, without limitation, centrifugation, or vacuum centrifugation.

The acids and bases that can be used are either organic or inorganic. Potentially suitable acids can be, but are not limited to, acetic acid, citric acid, formic acid, hydrochloric acid, lactic acid, phosphoric acid, phosphorus acid or sulfuric acid. Potentially suitable bases can be, but are not limited to, sodium hydroxide, potassium hydroxide, lithium hydroxide, Na₂CO₃, Ca(OH)₂ or NH₄OH.

Liquids with pH around neutral can also be used. Neutral solutions can be buffer or saline solutions, or water. Solutions that can be used in the process include but are not limited to organic and inorganic salt solutions. Examples are well known and include, but are not limited to, NaCl, NaF, NaBr, KCl, KF, KBr, sodium phosphate, sodium acetate, potassium citrate, ammonium acetate, sodium lactate, potassium phosphate, other phosphates and other salts. Mixtures are also contemplated, such as in phosphate buffered saline (abbreviated PBS). PBS is a commonly used buffer solution containing sodium chloride, sodium phosphate, and, in some formulations, potassium chloride and potassium phosphate.

Tissue slurries may be mixed with demineralized bone matrix (DBM) to make the molded tissue of the present application. In some embodiments, the DBM may represent 0-99% of the composition. In some embodiments, the DBM may represent 10-90% of the composition. In some embodiments, the DBM may represent 20-80% of the composition. In some embodiments, the DBM may represent 30-70% of the composition. In some embodiments, the DBM may represent 40-60% of the composition. In some embodiments, the DBM may represent 50-99% of the composition. In some embodiments, the DBM may represent 60-90% of the composition. In some embodiments, the DBM may represent 70-80% of the composition. In some embodiments, the DBM may represent 0-50% of the composition. In some embodiments, the DBM may represent 10-40% of the composition. In some embodiments, the DBM may represent 20-30% of the composition.

The molded tissue may be a graft unto itself and, when used alone, its advantageous properties can be fully leveraged, such as compressibility, conformability, and ability to be dehydrated and rehydrated. Also, the molded tissue may serve as a molded tissue component of a multi-component graft. Multi-component grafts may, for example, be unitary grafts that are made in composite for utilizing a bone component and a molded tissue component, or may be assembled from multiple bone components and molded tissue component(s) into an assembled graft.

DBM particles are typically from 150 microns to 900 microns, more typically from 250 microns to 800 microns, most typically from 400 to 500 microns. After mixing the tissue slurry and DBM, the mixture is molded and dehydrated. Dehydration can be performed via air drying, lyophilization or other methods known in the art.

It is contemplated that osteoinductive or active DBM may be used in bone repair, bone regeneration or bone healing applications. It is also contemplated that non-inductive or inactive or deactivated DBM may be used in applications not involving bone repair such as soft tissue repair, hernia repair or other non-bone applications. Osteoinductivity may be determined by methods known in the art including in-vivo or in-vitro assays. Osteoinductive DBM may be inactivated or made non-inductive by inactivation methods known in the art, including chemical and thermal methods.

The implants of the present application, when utilized in bone or hard tissue repair, replacement or treatment, may include demineralized bone matrix (DBM), bone chips, bone powder, calcium containing compounds, bone morphogenetic proteins (BMPs), growth factors (GFs), or other bone growth promoting, osteoconductive or osteoinductive elements. The implants of the present application, when utilized in soft tissue or non-bone repair, replacement or treatment, may be used without bone growth promoting, osteoconductive or osteoinductive elements and may be used with growth factors, cells, proteins, non-inductive or inactive or deactivated DBM or other elements known to promote soft tissue regeneration, repair or healing.

There are a number of different methods of producing DBM from various starting materials. DBM is commonly prepared by acid extraction of bone, resulting in loss of most of the mineralized components but retention of collagen and noncollagenous proteins. Other methods include alkaline extraction of bone. Any process known to those familiar with the technology can be used. Some specific processes are discussed below merely for exemplary purposes.

In one method of demineralization, a section of source bone is treated to remove soft tissue, including marrow and blood, and is then perforated to form a multiplicity of holes of desired size, spacing, and depth. The perforated bone section is then immersed and demineralized in an acid bath (e.g., 0.6 Normal (N) hydrochloric acid (HCl)), and is further treated in a defatting solution to remove or substantially reduce remaining marrow and intra-matrix cells. Following the perforating and defatting steps, the grafts can be freeze-dried and stored in sterile bags at conventional room temperature for periods of up to one year and perhaps longer prior to implantation or prior to use in the present methods of making a carrier, osteoinductive putty, or other implantable compositions.

In another method of making DBM, DBM is prepared by first removing all soft tissue and washing the bones in sterile deionized water. The cleansed bones are then extracted in a chloroform-methanol mixture, dried overnight, milled, sieved and decalcified in 0.6 N HCl for three to four hours. The resultant powder is rinsed with sterile deionized water to bring the pH to 3.5 or above and then lyophilized.

In yet another method of making DBM, DBM is prepared by soaking the bone segments for several minutes in a container with enough sterile ethanol to cover the tissue. The bone segments are milled and placed in a sieve. The milled bone material is cleaned with hydrogen peroxide, removed and rinsed with sterile water. The rinsed bone powder is added to sterile ethanol. The bone powder is then dried. The dried bone powder is transferred to the demineralization process. The bone powder is mixed with 0.6 N HCl until most of the mineral content is removed from the bone. The bone powder can be left for a longer period of time to fully demineralize the bone powder.

Additionally additional osteoconductive or biologically active materials such as mineralized bone matrix, cortical cancellous chips (CCC), crushed cancellous chips, tricalcium phosphate, hydroxyapatite, biphasic calcium phosphate, muscle fibers, collagen fibers, growth factors, antibiotics, cells, or other additives can be utilized in the grafts of the present application. When CCC is used, the mean particle size is typically 0.5 mm to 5 mm, more typically 1 mm to 3 mm, and even more typically from 1.5 mm to 2.5 mm.

The grafts of the present application show properties such as absorption, compression, expansion, resiliency and shape retention. Absorption refers to the ability to absorb fluid. Fluids can be, for example, water, blood, blood components, saline, platelet rich plasma (PRP), bone marrow aspirate (BMA) or combinations thereof. Absorption may be different for a rehydrated vs. a dehydrated implant and for different implant states. Absorption ranges can be approximately 4-6× for water and 2-4× for blood (vs. original graft weight). Absorption of blood or bodily fluids by the implants of the present application may be improved by first hydrating with water or saline, then compressing to force out some of the fluid volume before a second, third or later hydration with blood, PRP or BMA, for example.

Compression refers to the ability of a graft to undergo a reduction in volume or size under a force such as pressure applied manually or through some mechanism or machine. Implants of the present application may compress down to about 99% to about 10% of their initial height or volume under mild manual manipulation such as pressing down gently with one or more hands or fingers across at least a portion of an implant surface. In certain embodiments, implants of the present application may be compressed from about ½ to about ¼ of their original thickness or volume under focused manual manipulation such as squeezing tightly between two hands or fingers or by one hand or finger against the top of an implant supported by a solid surface beneath, or rolling or folding while compressing by hand. In certain embodiments, implants of the present application may be compressed from about ¼ to about 1/10 of their original thickness or volume under strongly focused manual manipulation such as rolling tightly into a compressed spiral while squeezing the entire implant to increase compression, or under a mechanized compression such as rolling and compressing within an arbor or rolling and compressing by hand then feeding into an arbor or container. In certain embodiments, implants of the present application may be compressed to less than about ¼ or less than about 1/10 of their original thickness or volume under focused mechanized manipulation such as compressing through fixed rollers then capturing the compressed implant and further folding or rolling while compressed, or such as rolling or folding while simultaneously compressing. The grafts of the present application may be compressed in a uniform or non-uniform manner along a single or multiple axis orientation. For example a graft may be compressed vertically and horizontally at the same time with the same or different compression ratios applied to each end of the graft. A graft may also be compressed radially and longitudinally at the same or at different times, such as by the passage of a cylindrical, spherical, rectangular, irregular or prismatic molded implant through a tapered, conical, or reduced diameter tube with longitudinal pressure forcing or extruding the graft from the end of the tapered tube.

Expansion refers to the ability to expand after compression; e.g. the graft expands with rehydration or following a compression to at least its original thickness. Expansion may be triggered by rehydration or may occur in an already rehydrated graft following compression and release of the compressing force. Expansion is typically associated with and more prevalent among hydrated grafts vs. dehydrated grafts. In some cases dehydrated grafts may undergo negligible expansion. In some cases dehydrated grafts may be stored without significant physical restrictions on their shape, form or size. In some cases dehydrated grafts may resist expansion until after rehydration. In some cases grafts may be stored in a restrictive container, mold or conveyance following a second dehydration in a compressed state before being rehydrated within the container, mold or conveyance prior to surgical application. In some cases the graft may be passed into the surgical site prior to rehydration such that expansion or shape retention or both are observed at the surgical site following delivery or initial implantation in a patient in a dehydrated state. In some cases the graft may be rehydrated before or just before delivery to the surgical site such that expansion or shape retention or both are observed at the surgical site following delivery or initial implantation in a patient in a compressed or partially compressed and hydrated or partially hydrated state. Resiliency refers to the ability to retain shape after rehydration and compression. It also refers to the ability of the material to resist tearing or shearing. This can also be called cohesion.

Shape retention refers to a property of the material to maintain its original shape or the shape of the mold/void after hydration. The graft maintains a desired shape and does not dissolve away during surgery or prematurely during healing. Shape retention includes swelling that may increase overall volume, but retains comparable dimensional ratios. Shape retention further includes the property of returning to an original shape or to about an original shape, or to substantially about an original shape following a compression and optionally following a dehydration and rehydration of the graft. The grafts also show good regeneration and incorporation into the patient, with lack of appreciable inflammation.

The grafts of the present application are flexible, yet cohesive; and are easy to cut, shape and mold. The grafts are compressible, conformable, and hydratable. Compressible refers to a reduction in volume with applied pressure (in the case of the grafts of the application, this includes the potential to return to the original volume). Conformable refers to the property of the grafts to expand (upon rehydration) to adapt to the contours of an implant site. Hydratable refers to the property of the grafts to be able to be hydrated with several different fluids, after being first presented in a dehydrated form. Suitable fluids can be, but are not limited to, water, blood, blood components, saline, platelet rich plasma, bone marrow aspirate or combinations thereof.

The grafts of the application have shape retention and can be provided in a compressed form for minimally invasive surgical methods. Minimally invasive procedures, which include but are not limited to laparoscopic (also called keyhole), arthroscopic, endoscopic and thoracoscopic surgery, use methods to reduce the damage to human tissue when performing surgery. The procedure (surgery) is carried out by entering the body with the smallest damage possible. The incision(s) used to perform the procedure are much smaller than in traditional surgeries. In one embodiment, a surgeon makes several small incisions (about ¼ inch to one inch in length) which are used as portals, optionally thin tubes (e.g. trocars) are used. Specialized instruments and optionally a camera, sensor or other visualization apparatus are placed through the trocars or portal to perform the procedures. In other embodiments, a slightly larger incision may be needed. In other embodiments, no incisions and/or trocars are necessary. Other minimally invasive surgical procedures can be performed almost exclusively through a single point of entry (only one small incision) or through a naturally occurring point of entry. Minimally invasive surgical procedures include delivery of the graft through the use of endovascular catheters, including, for example, percutaneous transluminal coronary intervention and neurovascular aneurysm coiling. Minimally invasive surgical procedures contemplated by this application also include access through a body orifice including, for example, endoscopy, cystoscopy and nasopharyngeal access.

Minimally invasive procedures allow for less blood loss, quicker recovery, shorter hospital stays, less scarring and less postoperative pain for the patient. Procedures in the areas of abdominal, heart, digestive, colon, ENT (ear, nose and throat), lung and others can be minimally invasive. Other candidates for minimally invasive procedures are bariatric or gynecological surgeries. Robotic surgery may also be used in minimally invasive procedures. This technology gives doctors increased precision, flexibility and control.

Specifically, the grafts can be molded and dehydrated into a preferred shape for implantation, and then compressed and dehydrated into a form that is smaller in at least one dimension. The graft then “bounces back” to its original, preferred shape after implantation. This allows for the larger size grafts to be inserted into a surgical site through a minimally invasive access portal. Preferred shapes or configurations are a square, rectangle, strip, circle, triangle or oval. Irregular, asymmetric, symmetric, geometric, custom, anatomical, and patterned shapes are contemplated. Shapes having specific properties related to compression and expansion are contemplated, for example a nested or fractal pattern shape which may be folded or compressed into itself is contemplated. Optionally the implants (grafts) are packaged and sterilized.

In one embodiment, the graft may also be coated or encapsulated. This allows for more facile entry into a surgical site (e.g. delivery via a catheter). This may also allow for staged or controlled release and/or delivery of the graft. The coating or encapsulation materials could be another layer of the biological tissue graft material itself, multiple layers of the biological tissue or other synthetic or natural materials. For example, the graft may be coated with one or more coatings that have certain release profiles (e.g. dissolution rates). Another example would be the encapsulation of the graft within one or more materials that have certain release profiles. Release profiles can be chosen to allow for staged physical and/or chemical access to the graft during certain surgical procedures and/or after the graft is placed at a surgical site. A non-limiting example of encapsulation material would be a gelatin capsule. The coating or encapsulation may occur during or after the graft is made into a form that is smaller in at least one dimension (e.g. during or after the compression and/or dehydration steps). In one non-limiting example an implant of the present invention is coated in a layer of a first coating material of the same or a similar material formulation as that of the implant itself, forming one or more layers. The implant is then optionally further folded or compressed to a final implant configuration and coated with a second material, that is the same or different from the first coating material. This coated implant could then be encapsulated within a gelatin capsule prior to final packaging and delivery to a surgical site for implantation. The coatings and encapsulations may be designed so that one layer dissolves or breaks up in minutes to hours post implantation, a second layer lasts hours to days post implantation, and a third layer remains in place for days to weeks post implantation. Each layer may allow or control the timing of a physical transformation such as unfolding, swelling, conforming or filling a void; and/or timing of a fluid or other material's access to the implant, a chemical or biochemical access to the implant, or a cellular, protein or other biological agent's access to the implant. Further, each coating layer or encapsulant may provide protection or separation of the implant from these or other elements at the surgical site.

In one embodiment, the present application is directed to a dehydrated, packaged and sterilized graft in the form of a compressed sheet for implantation into a human patient. In this embodiment the implant is compressed into a first configuration suitable for passage into a human patient and is further capable of opening and expansion to a second configuration suitable for application to a surgical site within the human patient. The graft has an initial dehydrated thickness and an initial dehydrated shape and a hydrated thickness greater than that of said initial dehydrated thickness. In this embodiment the second configuration is larger in at least one dimension than the first configuration. In a method of surgery using a minimally invasive access portal having a cross sectional area, the compressed sheet will fit through the portal, but upon rehydrating and expanding the graft at the surgical site within the patient, the graft covers an implant area that is larger than said cross sectional area of the minimally invasive access portal. Optionally included are steps of positioning or fixing the implant in place at the surgical site to create a fixed implant. The expanded (or rehydrated) implant has a fixed unitary shape which is larger than that which could have passed through the minimally invasive access portal.

The compression is preferably achieved via any action that decreases the size of the graft in at least one dimension, while allowing at least one other dimension to remain essentially unchanged. For example, the actions of rolling, folding, spiraling, winding or crumpling are contemplated. The compression can occur in a controlled, regular pattern, or in an uncontrolled random (irregular) way. The compression can occur in one or more dimensions. Expansion of the graft is due, at least in part, to contact with a hydration fluid (rehydration).

In one embodiment, the present application is directed to a unitary biological implant for implantation into a human patient, consisting essentially of human tissue components in a dehydrated state. In this embodiment the implant has a first thickness and a first characteristic width immediately following an initial dehydration. In this embodiment the implant is rehydrated, then compressed to a second thickness less than about ¾ of the first thickness and rolled into a shape having a second characteristic width or rolled diameter less than about ½ of the first characteristic width, to create a compact or compressed implant suitable for passage through a minimally invasive portal into a surgical site. In some embodiments the implant undergoes a second dehydration in a rolled configuration, and may be rehydrated either before or after passage through the minimally invasive portal. Whether rehydrated before or after passage into the surgical site, in certain embodiments the implant is able to unroll to substantially about the first characteristic width and to swell back to at least substantially about the first thickness within the patient without substantial cracking, tearing or damage to the unitary biological implant. In other embodiments, the compression is to less than substantially about ½ or ¼ of the first thickness and/or the diameter less than substantially about ¼ or ⅛ of the first characteristic width and compression and rehydration occur substantially without tearing the implant.

In one embodiment, the present application is directed to a method of surgery on a human patient, by providing an allograft unitary implant having a first shape, cutting the allograft unitary implant to a desired shape, rehydrating the allograft unitary implant with a first rehydration fluid to create a first rehydrated implant. Then the first rehydrated implant is worked, substantially without cracking or tearing, into a second shape which is compressed in volume by at least about half relative to the first shape. The rehydrated implant is placed at a surgical site within a human patient. Before placing the graft at the surgical site, it is optionally further hydrated with a second rehydration fluid to create a second rehydrated implant. The second rehydration fluid may be the same or different than the first rehydration fluid. Preferably the allograft unitary implants are made from essentially of human tissue components.

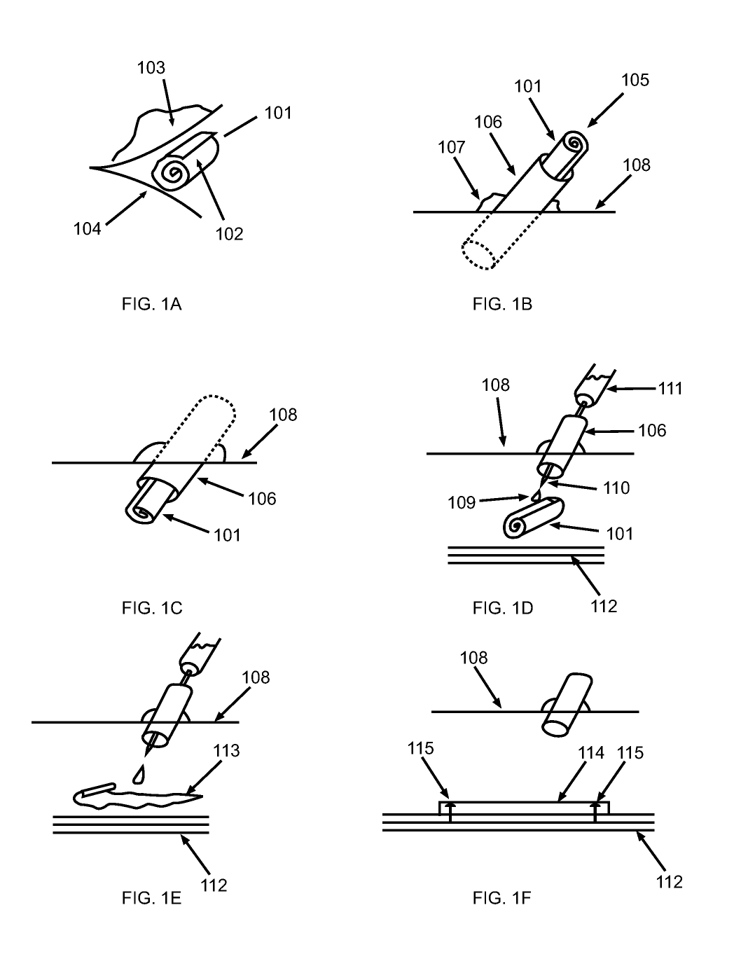

FIGS. 1A-1F show certain stages in one surgical application of a minimally invasive procedure using a rolled compressed dehydrated packaged sterilized molded tissue graft comprising demineralized bone together with a human tissue slurry.

FIG. 1A shows a molded tissue graft 101 comprising demineralized bone matrix together with a human tissue slurry, having a rolled compressed configuration 102, contained within a package including a sterile barrier peel pouch 103 and backing 104.

FIG. 1B shows a molded tissue graft 101, being inserted in direction 105 through a minimally invasive portal 106, into a minimally invasive surgical opening 107 into a patient body 108.

FIG. 1C shows a molded tissue graft 101, passing through a minimally invasive portal 106, into a patient body 108.

FIG. 1D shows a molded tissue graft 101, having passed through a minimally invasive portal 106, into a patient body 108, and lying adjacent a target surgical tissue 112, being hydrated by a hydration fluid 109 through a hydration needle 110 being supplied by a hydration source 111 from outside the patient body 108.

FIG. 1E shows a rolled compressed molded tissue graft unrolling to a partially open configuration 113 following hydration inside a patient body 108, and adjacent a target surgical tissue 112.

FIG. 1F shows a rolled compressed molded tissue graft essentially completely unrolled to an open configuration 114 following hydration inside a patient body 108, and affixed to a target surgical tissue 112 by fixation means 115.

Referring next to FIG. 2, another embodiment of a compressed dehydrated packaged sterilized molded tissue graft comprising demineralized bone together with a human tissue slurry 201 can be seen. The compression is performed by folding, the compressed graft having minimum bend radii 202 and compressed thickness 203, and being captured within a sterile barrier packaging having a backing 204, a top cover 205, a first seal 206, a second seal 207 and a peal opening 208.

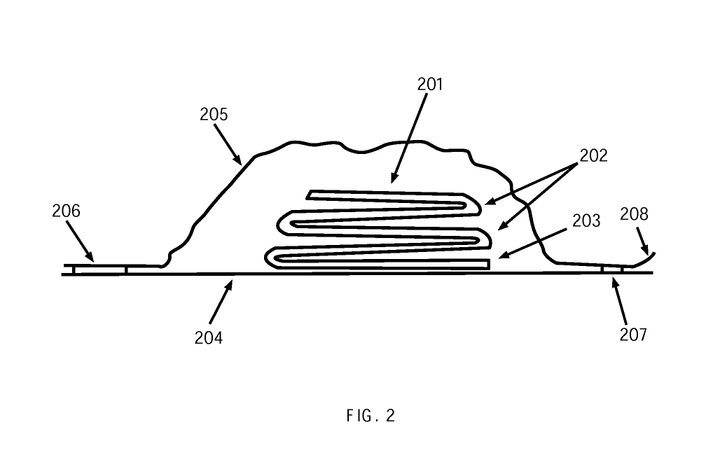

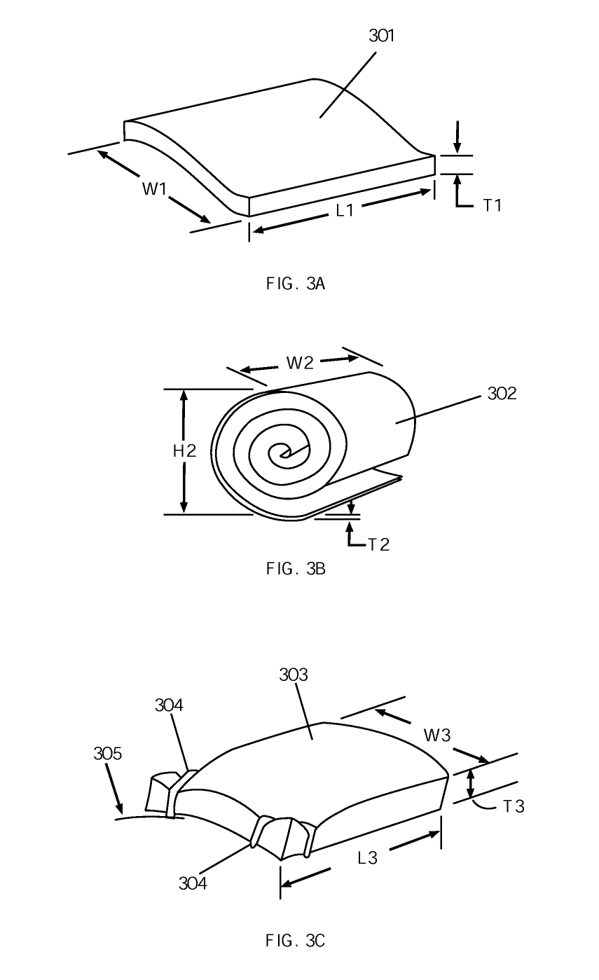

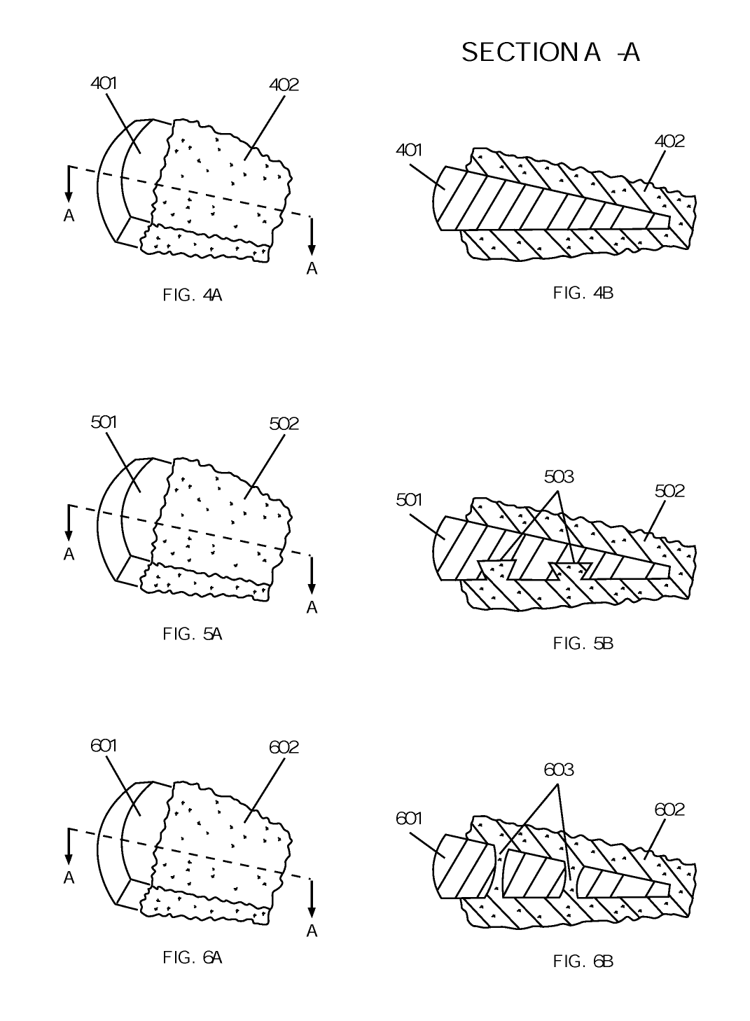

FIGS. 3A-3C show a molded tissue graft in (a) initial solid dehydrated state 301 with initial width W1, initial length L1, and initial thickness T1, (b) rehydrated, compressed, rolled and finally dehydrated state 302 with rolled width W2, rolled height H2, and rolled thickness T2, and (c) secondarily rehydrated, expanded and unrolled state 303 with final width W3, final length L3, and final thickness T3 with fixation 304 at a surgical site 305 within a patient.

The grafts of the present application can also be used as a component in bone block implants. The properties of the graft (capability to be molded) allows for incorporation of the graft material with a hard, bony component. In one such embodiment, a unitary allograft bone block implant is formed that contains a block of cortical bone, cancellous bone or both; and a molded tissue component which is integrated into the block. Optionally, the implant also has a compression zone formed by the molded tissue component, outside of the bone graft and adjacent to the machined outer surface. This compression zone exhibits expansion upon hydration of the implant at the surgical site sufficient to fill voids within adjacent tissues of the human patient.

FIG. 4 shows two views of a composite tissue wedge of human bone 401 together with a molded tissue component 402.

In one embodiment, the bone block is a wedge or trapezoid and the molded tissue component is formed along at least one outer surface of the wedge or trapezoid. In other embodiments, the bone block is in the shape of a ring and the molded tissue component can fill all or some of the space inside.

In another embodiment, the bone block is a wedge and the molded tissue component is formed along at least one outer surface of the wedge and has a homogenous transition between an internal portion contained within the pores of the unitary allograft bone wedge and an external portion having an initial thickness adjacent the unitary allograft bone wedge; wherein the internal portion anchors the external portion to the implant during implantation and wherein the external portion expands to at least about 1.1×, optionally at least about 1.25×, optionally at least about 1.5×, optionally at least about 1.75×, optionally at least about 2× its initial thickness following implantation, rehydration or both. The bone block may have discreet retention features created along one surface, for example, depressions that can be filled with the molded tissue component. In this or in other embodiments the molded tissue may cover a portion of one or more sides of the bone block, may stop short of one or more edges of the bone block, may reach to or stop at one or more edges of the bone block, may wrap around one or more corners of the bone block, and may totally or partially encapsulate one or more regions of the bone block with or without specific areas left uncovered for the application of surgical instruments, implant pathways, interface with patient anatomy, or for surgeon access to the bone block.

Referring next to FIG. 5, a composite tissue wedge of human bone 501 is shown together with a molded tissue component 502 having discreet retention features 503 along one surface.

The bone block may also contain holes or passageways that extend through the graft that can be spanned by the molded tissue component. In such embodiments, the passageways allow communication between two surfaces and provide a physical mechanism to hold the molded material in place on either side of the bone block. FIG. 6 shows a composite tissue wedge of human bone 601 covered in an all human tissue matrix carrier 602 having passageways 603 in communication between two surfaces.

In another embodiment, the present application is directed to a dehydrated composite biological implant useful for surgical implantation at a surgical site within a human patient comprising a rigid bone graft of cortical bone, cancellous bone or both, having at least one machined outer surface. A machined outer surface may include a non-native bone surface and may be finished by a milling machine or lathe, by a grinder, rasp or file, or by a had tool, drill or saw. In this embodiment the implant also has at least one layer of molded tissue component that is osteoconductive, compressible, conformable and hydratable. The molded tissue component is incorporated into the pores of the bone graft at the outer surface such that the tissue matrix is incorporated into the bone graft and covers at least a portion of the machined outer surface. In one embodiment, the molded tissue component is frozen and lyophilized in place along at least a portion of at least one outer surface of the bone block. In another embodiment, the molded tissue component covers at least two sides of said bone block and substantially fills a passageway between at least two of said at least two sides.

The grafts of the present application can also be used as a component in assembled bone implants to assist in adhesion of components, fill voids in the assembly and/or to provide size options or flexibility to the assembly. The properties of the graft (capability to be molded) allows for use of the graft material together with a hard, bony component to achieve, for example, compression, expansion and conformability not normally available with bone grafts. In one embodiment, the present application is directed to an assembled bone allograft implant comprising two or more substantially planar segments of cortical bone, cancellous bone or both joined together by one or more pins of cortical bone which also contains at least one molded tissue component which is osteoconductive, compressible, conformable, hydratable and which is sandwiched between at least two substantially planar segments of cortical bone, cancellous bone or both. The molded segment is formed from a tissue slurry of substantially of non-bone origin together with demineralized bone matrix (DBM).

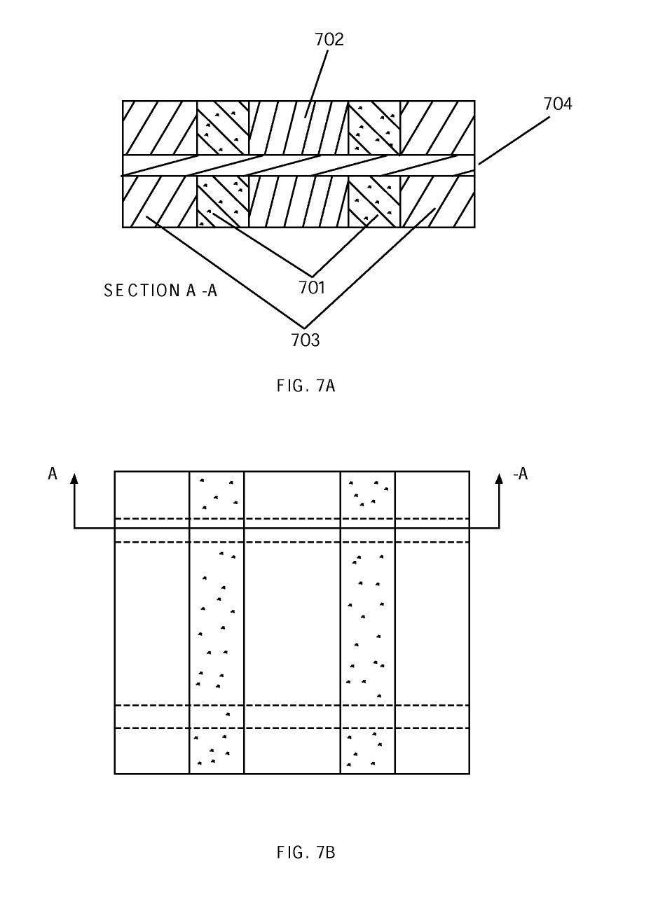

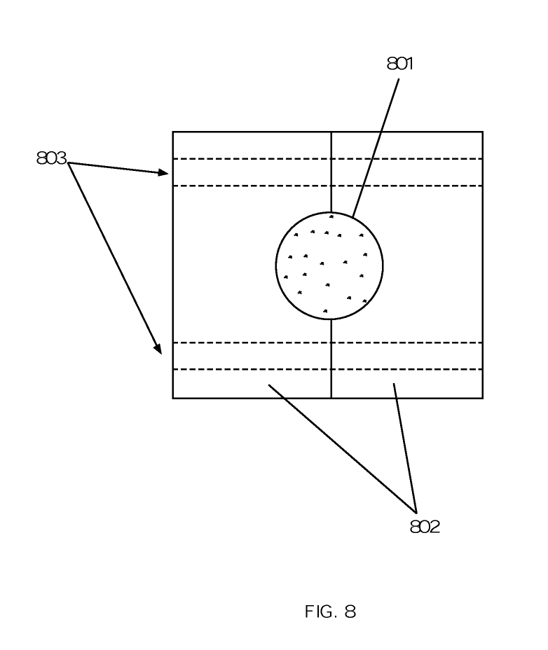

FIG. 7 shows a composite bone implant having cortical bone 703, cancellous bone 702 and molded tissue components 701 assembled to form a layered implant structure fastened together by a human bone pin 704. The pin can be made from cortical or cancellous bone.

In one embodiment, an assembled biological implant useful for surgical implantation at a surgical site within a human patient can be made having at least one segment of the assembly made from a molded tissue component. The molded tissue component is formed from a tissue slurry of substantially of non-bone origin together with demineralized bone matrix (DBM). The implant can also have at least one bone component of cortical bone in direct contact with the molded segment of human tissue matrix and optionally having at least one machined outer surface. In this embodiment the implant may also have at least one bone pin securing the molded component to the bone component.

FIG. 8 shows a composite bone implant having a molded tissue component 801 molded between and completely surrounded by two cortical bone components 802 and held together by bone pins 803.

In assembled bone implants, the bone components can be in the shape of a plank, square, rectangle or trapezoid. There can be multiple bone components, in some embodiments two bone components, in others 3, 4, 5, 6, 7, 8, 9, 10 or more bone components can be utilized. The bone components can be cortical or cancellous bone, or both and can be optionally fully or partially demineralized. One such assembled biological implant useful for surgical implantation at a surgical site within a human patient can be made having at least one segment of the assembly made from a molded tissue component. The molded tissue component is formed from a tissue slurry of substantially of non-bone origin together with demineralized bone matrix (DBM). The implant can also have at least one bone component of cancellous bone in direct contact with the molded segment of human tissue matrix and optionally having at least one machined outer surface. The implant also may have at least one rigid bone graft of cortical bone in direct contact with the molded tissue component or with the cancellous bone or with both, and optionally having at least one machined outer surface. In this embodiment the implant may also have at least one bone pin securing the molded segment to the bone grafts or pinning the molded segment between two or more bone segments.

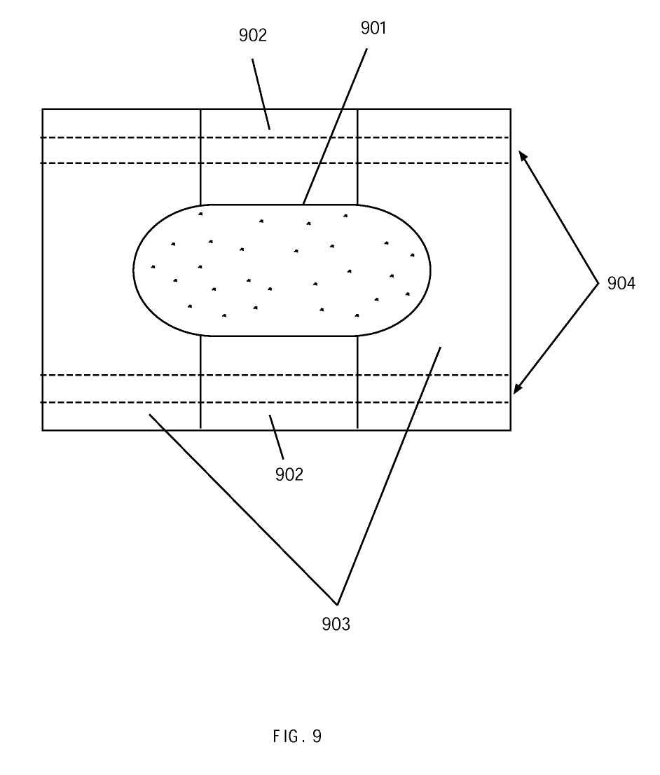

Various assembled grafts are shown in FIGS. 9-12. Specifically, FIG. 9 shows a composite bone implant having a molded tissue component 901 molded between and contacting at least 4 distinct pieces of cortical bone including two center pieces 902 and two end pieces 903 held together by bone pins 904. The molded tissue component can form a pathway between the top and bottom surfaces of the implant.

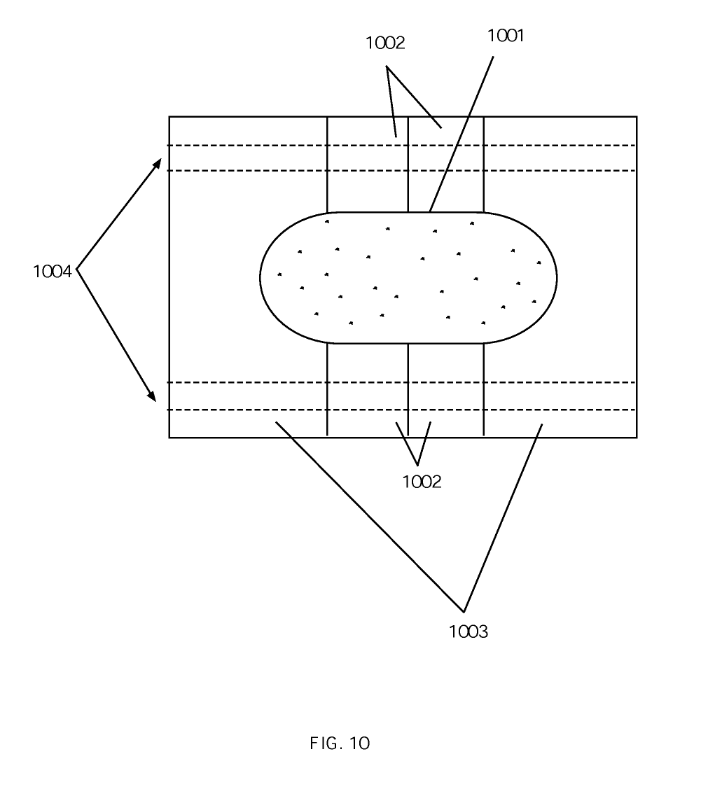

FIG. 10 shows a composite bone implant having a molded tissue component 1001 molded between and contacting at least 6 distinct pieces of cortical bone including four center pieces 1002 and two end pieces 1003 held together by bone pins 1004. The molded tissue component can form a pathway between the top and bottom surfaces of the implant.

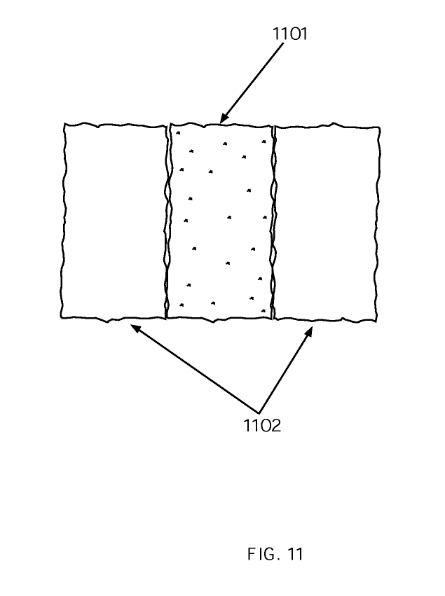

FIG. 11 shows a composite bone implant having a molded tissue component 1101 sandwiched between and contacting at least 2 cancellous bone planks 1102. The molded tissue component can form a pathway between the top and bottom surfaces of the implant.

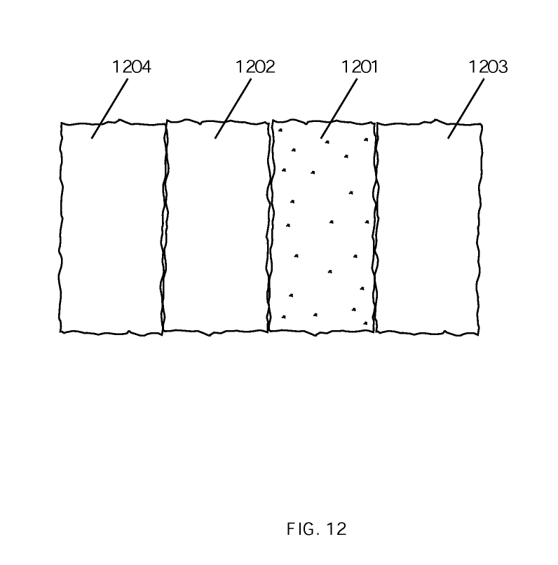

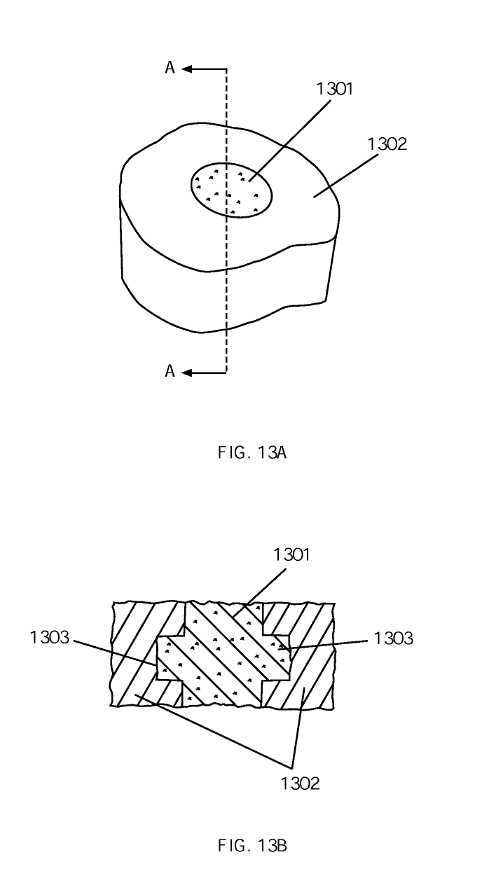

FIG. 12 shows a composite bone implant having a molded tissue component 1201 sandwiched between and contacting at least 1 cancellous bone plank 1202 and at least 1 cortical bone plank 1203 and not in contact with another cortical bone plank 1204. The molded tissue component can form a pathway between the top and bottom surfaces of the implant.

In other embodiments, additional composite grafts comprising a rigid bone graft component of cortical bone, cancellous bone or both are contemplated. Bone components may be, but are not limited to shapes such as plugs, rings, planks or struts. The bone components may optionally have at least one machined outer or inner surface. The machining may be used to create features (recesses, holes, channels, etc.) for the molded tissue component to fill, and/or for purposes of shaping the graft itself, for ease in insertion during surgery and/or to resist migration or movement of the graft during implantation. In one embodiment, the bone component contains one slot, groove or hole to allow for increased penetration of the molded tissue component. In one embodiment, the molded tissue component is frozen and lyophilized in place along at least a portion of at least one outer surface of the bone block. In another embodiment, the molded tissue component covers at least two sides of said bone block and substantially fills a passageway between at least two of said at least two sides.

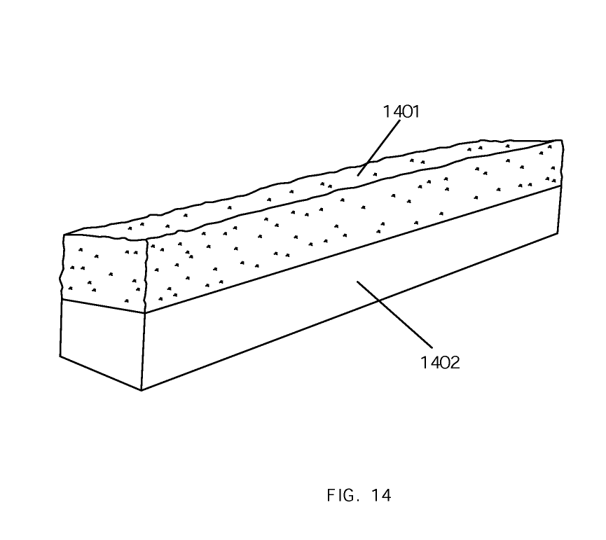

Various composite grafts are shown in FIGS. 13 and 14. FIG. 13 shows a composite bone implant having a molded tissue component 1301 molded into a machined recess within a bone plug 1302, with an inner retention feature 1303 to aid in retention of the molded tissue component within the machined recess. A molded tissue component may similarly be molded into a natural recess, tunnel or canal existing in a bone graft, with or without the aid of one or more retention features. Retention features may be regular or patterned, and of various shapes, sizes, locations and number useful to retain a molded component within a given bone graft. In some cases a retention feature may not be required, or may be a naturally occurring feature. FIG. 14 shows a composite allograft cortical bone strut implant with a molded tissue component 1401 covering one side of the cortical bone strut 1402. A bone strut with surface features, roughenings, protrusions or depressions may be used to aid in bonding or retention of the molded tissue component. The bone strut may be completely or partially encapsulated by the molded tissue. Similarly a molded tissue component may be added to other structures such as a ligament or tendon graft, a spinal implant, a conventional bone allograft construct, one or more bone blocks of a bone-tendon-bone or bone-tendon graft, or partially or fully surrounding or encapsulating the tendon or tendon and bone block or blocks of a bone-tendon-bone or bone-tendon graft.

### EXAMPLES

### Example 1

### Creation of a Minimally Invasive Tissue Implant

A molded tissue implant may be prepared through acid treatment of human tissue material to create a slurry which may then be rinsed and mixed with demineralized bone matrix (DBM). Suitable processes for creating a tissue slurry are disclosed in assignee's U.S. Pat. No. 7,001,430, entitled “Matrix Composition for Human Grafts/Implants,” which issued to Mills et al., on Feb. 21, 2006; U.S. Pat. No. 7,131,994, entitled “Muscle-Based Grafts/Implants,” which issued to Mills, et al. on Nov. 7, 2006; and U.S. Pat. No. 7,883,541, entitled “Muscle-Based Grafts/Implants,” which issued to Mills et al., on Nov. 25, 2003, all of which are incorporated herein by reference in their entirety.

The DBM and human tissue slurry is poured into a mold about 25 mm wide by about 75 mm long by about 5 mm deep. The molded slurry is then frozen and lyophilized to create a dehydrated tissue sheet. The dehydrated implant is rehydrated by exposure to sterile water for about 10 minutes at ambient temperature. Following rehydration the implant is compressed and rolled by hand into a cylindrical spiral form of about 75 mm long by about 10 mm diameter, then stored within a cylindrical container, refrozen and lyophilized. Following this second dehydration, the implant is transferred to a second cylindrical container and sealed within a package for storage in a compressed, rolled, dehydrated state. Upon removal from the package, the implant is passed through a portal of about 10 mm in diameter then rehydrated with sterile saline and allowed to return to a shape and dimensions about the same as those observed in the original dehydrated tissue sheet.

### Example 2

### Rolled or Folded Sheet for Minimally Invasive Surgery

In one embodiment, a molded biological graft for implantation into a human patient may be formed from only human tissue components in a dehydrated state. In this embodiment the graft may be formed into a low, flat mold of about 0.5 mm to about 5 mm in thickness and about 10 mm² to about 500 mm² in surface area, frozen, lyophilized and removed from the mold. The mold may be round, rectangular, or of any shape to suit the surgical application. The dried implant may be rehydrated and then rolled or folded loosely or tightly, with optional compression, by hand or through use of a mandrel or winding or folding apparatus, into a first configuration suitable for passage into a human patient. The implant may be optionally dried in the rolled or folded state, further processed or sterilized and packaged in intermediate or final packaging before being delivered to a surgical site and then passed through a portal suitable for minimally invasive surgery before unfolding or unrolling, opening and expansion to a second configuration suitable for application to a surgical site within the patient. In this embodiment the second configuration is larger in width than the first configuration.

The implant may be placed at the surgical site and allowed to rehydrate from blood or other bodily fluids of the patient, or the implant may be rehydrated after entering the surgical site, but prior to fixation at the surgical site. The implant may also be rehydrated at the surgical site prior to implantation, or delivered to the surgical site in a hydrated state. The implant may be advantageously configured to a shape change, such as simple swelling or a more complex change in geometry, upon rehydration.

Alternatively, the implant may be rehydrated with one or more fluids before being rolled or folded loosely or tightly, with optional compression, into a first configuration suitable for passage into a human patient and then passed through a portal suitable for minimally invasive surgery before unrolling, opening and expansion to a second configuration suitable for application to a surgical site within the patient.

Alternatively, the implant may be rehydrated, further hydrated or secondarily hydrated with one or more fluids after being rolled loosely or tightly, with optional compression, into a first configuration suitable for passage into a human patient and then passed through a portal suitable for minimally invasive surgery before unfolding or unrolling, opening and expansion to a second configuration suitable for application to a surgical site within the patient.

### Example 3

### Compressible Unitary Implant

In one embodiment, a unitary biological implant for implantation into a human patient may be formed essentially of human tissue components in a dehydrated state. In this embodiment the implant may have a first thickness and a first characteristic width immediately following an initial dehydration. In this embodiment the implant may be compressed to a second thickness less than about ¾ of the first thickness and rolled into a shape having a second characteristic width less than about ½ of the first characteristic width, to create a compact or compressed implant suitable for passage through a minimally invasive portal into a surgical site and able to unroll to substantially about the first characteristic width and to swell back to at least about the first thickness within the patient without substantial cracking, tearing or damage to the unitary biological implant.

### Example 4

### Compressible Unitary Implant

In one embodiment, a dehydrated composite biological implant useful for surgical implantation at a surgical site within a human patient, consisting essentially of human tissue, and having at least one molded tissue component, formed of a liquid matrix substantially of non-bone origin dried in a mold and having at least one molded outer surface may be formed. In this embodiment the implant may have at least one bone component of cancellous bone in direct contact with the molded tissue component and having at least one machined outer surface. In this embodiment the implant also may have at least one rigid bone component of cortical bone in direct contact with the molded tissue component or with the bone component of cancellous or with both, and having at least one machined outer surface. In this embodiment the implant also may have at least one bone pin securing the molded segment to the bone components.

### Example 5

### Assembled Composite Implant

In one embodiment, an assembled bone allograft implant may be made from two or more substantially planar segments of cortical bone, cancellous bone or both joined together by one or more pins of cortical bone which has a layer of comminuted, acid treated, and dried substantially non-bone human allograft tissue which is osteoconductive, compressible, conformable, hydratable and sandwiched between at least two substantially planar segments of cortical bone, cancellous bone or both.

### Example 6

### Expanding Soft Surfaced Bone Wedge Implant

In one embodiment, an unitary allograft bone wedge implant may be made with a wedge of cortical bone, cancellous bone or both, which has a layer of comminuted, acid treated, and dried substantially non-bone human allograft tissue which is osteoconductive, compressible, conformable, hydratable and formed along at least one outer surface of the unitary allograft bone wedge. The added layer may be configured to have an homogenous transition between an internal portion contained within the pores of the unitary allograft bone wedge and an external portion having an initial thickness adjacent the unitary allograft bone wedge; wherein the internal portion anchors the external portion to the implant during implantation and wherein the external portion expands to at least about one and one tenth to one and one half times its initial thickness following implantation, rehydration or both.

### Example 7

### Multiple Zone Implant

In one embodiment, a dehydrated composite biological implant useful for surgical implantation at a surgical site within a human patient, may be made essentially of human tissue, and comprising a rigid bone component of cortical bone, cancellous bone or both, having at least one machined outer surface. In this embodiment the implant also may have at least one layer of a molded tissue component that osteoconductive, compressible, conformable and hydratable incorporated into the pores of the bone component at the outer surface such that the molded tissue component is incorporated into the bone component and covers at least a portion of the machined outer surface. In this embodiment the implant also may have a compression zone formed by the molded tissue component, outside of the bone component and adjacent the machined outer surface which exhibits expansion upon hydration of the implant at the surgical site sufficient to fill voids within adjacent tissues of the human patient.

### Example 8

### Filled Assembled Implant

A dehydrated composite biological implant useful for surgical implantation at a surgical site within a human patient was made essentially of human tissue, including an assembled rigid bone graft of cortical bone having machined outer surfaces and a machined inner opening from top surface to bottom surface. In this embodiment the implant also contained one layer of a molded tissue component that is osteoconductive, compressible, conformable and hydratable incorporated into the portal of the bone graft by molding the graft into the machined inner opening of the bone grafts before freezing and then dehydrating the implants. The frozen molded tissue component filled the inner opening from top to bottom surface of the implant, and exhibited mild shrinkage upon freezing and lyophilization. The observed shrinkage was reversed upon rehydration in a sterile solution. The molded tissue component was found to retain shape and position within the inner opening both pre and post rehydration under manipulation and pressure by hand and using tweezers. The molded tissue component was found to be more resilient and resistant to force following rehydration.

### Example 9

### Method of Minimally Invasive Surgery

In one embodiment, a method of surgery on a human patient is contemplated, comprising the steps of (a) providing an allograft unitary implant consisting essentially of dehydrated human tissue components in the form of a rolled compressed sheet, and (b) providing a minimally invasive access portal into a surgical site within a human patient, and (c) passing the implant through the minimally invasive access portal, and (d) providing a hydration fluid in contact with the implant to create a rehydrated implant, and (e) allowing or causing the implant to unroll and expand upon contact with the fluid to create an expanded implant, and (f) positioning or fixing the implant in place at the surgical site to create a fixed implant, wherein following steps (a)-(f) above, the allograft implant has a fixed unitary shape which is larger than that which could have passed through the portal.

### Example 10

### Method of Minimally Invasive Surgery

In one embodiment, a method of surgery on a human patient is contemplated, comprising the steps of (a) providing an allograft unitary implant consisting essentially of dehydrated human tissue components in the form of a rolled compressed sheet having an initial dehydrated thickness measurable across the sheet and an initial dehydrated shape prior to bring rolled, and (b) rehydrating the allograft unitary implant, and (c) passing the allograft unitary implant through a minimally invasive access portal having a cross sectional area into a surgical site within a human patient, and (d) unrolling and rehydrating the allograft unitary implant at the surgical site within the human patient, and (e) positioning the allograft unitary implant at the surgical site covering an implant area at least about twice that of the cross sectional area of the portal having a final hydrated thickness at least about twice that of the initial dehydrated thickness substantially without tearing, splitting or rupturing the allograft unitary implant.

### Example 11

### Method of Minimally Invasive Surgery

In one embodiment, a method of surgery on a human patient is contemplated, comprising the steps of (a) providing an allograft unitary implant consisting essentially of dehydrated human tissue components and having a first shape, and (b) cutting the allograft unitary implant to a desired shape, and (c) rehydrating the allograft unitary implant with a first rehydration fluid to create a first rehydrated implant, and (d) working the first rehydrated implant substantially without cracking or tearing into a second shape which is compressed in volume by at least about half relative to the first shape and which results in loss of at least about half of the first rehydration fluid by volume, and (e) further hydrating the first rehydrated implant with a second rehydration fluid to create a second rehydrated implant, wherein the second rehydration fluid is not of the same composition as the first rehydration fluid, and (f) fixing the second rehydrated implant at a surgical site within a human patient.

### Example 12

### Compression of Rehydrated Molded Tissue Implant

A dehydrated molded human tissue implant was made with a length of about 15 mm a width of about 10 mm and a thickness of about 6.6 mm, all initial measurements taken with a hand held digital caliper in the dehydrated state following molding and lyophilization. This implant was cut to length and width from a larger molded implant of the same thickness. The implant was then rehydrated with deionized water, following which a first rehydrated thickness of about 6.9 mm across the surface of the implant was recorded. The implant was then compressed under manual pressure to a compressed thickness of about 2.1 mm wherein the implant lost a portion of the rehydration fluid. The implant was then released from compression, recovering to a thickness of about 4.1 mm. The implant was then fully rehydrated and observed to return to about the original rehydrated thickness of about 6.9 mm across the surface of the implant.

While the application has been described with reference to certain embodiments, it will be understood by those skilled in the art that various changes may be made and equivalents may be substituted without departing from the scope of the application. In addition, many modifications may be made to adapt a particular situation or material to the teachings of the application without departing from its scope. Therefore, it is intended that the application not be limited to the particular embodiment disclosed, but that the application will include all embodiments falling within the scope of the appended claims.

1. A biological graft, consisting essentially of human tissue components made by;
molding the graft into a first configuration and dehydrating the graft;
rehydrating the graft with a first rehydration fluid and compressing the graft into a second configuration suitable for passage into a human patient wherein said second configuration is smaller in at least one dimension than said first configuration; and
dehydrating, packaging and sterilizing the graft in said second configuration.

2. The graft of claim 1, wherein said first rehydration fluid is water, blood, blood components, saline, platelet rich plasma or bone marrow aspirate.

3. The graft of claim 1, wherein said graft is rehydrated from said second configuration with a second rehydration fluid.

4. The graft of claim 3, wherein said second rehydration fluid is water, blood, blood components, saline, platelet rich plasma or bone marrow aspirate.

5. The graft of claim 4, wherein said second rehydration fluid and said first rehydration fluid are different.

6. The graft of claim 4, wherein said second rehydration fluid and said first rehydration fluid are the same.

7. The graft of claim 1, wherein said graft expands back to said first configuration upon application to a surgical site within said human patient.

8. The graft of claim 1, wherein said first configuration is a square, rectangle, circle, triangle or oval.

9. A method of surgery on a human patient using the graft of claim 1, comprising:
providing said dehydrated, packaged and sterilized graft in the form of a compressed sheet having an initial dehydrated thickness and an initial dehydrated shape, and
passing said graft into a surgical site within said human patient through a minimally invasive access portal having a cross sectional area, and
rehydrating and expanding said graft at said surgical site within said human patient, and
positioning said graft at the surgical site covering an implant area that is larger than said cross sectional area of said portal, and
fully rehydrating said graft, thereby causing said graft to return to a hydrated thickness greater than that of said initial dehydrated thickness.

10. A method of surgery on a human patient, comprising:
providing an allograft unitary implant consisting essentially of human tissue components in a compressed dehydrated form, and
providing a minimally invasive access portal into a surgical site within said human patient, and
passing said implant through said minimally invasive access portal, and
providing a hydration fluid in contact with said implant, wherein
said implant expands following contact with said hydration fluid to create an expanded implant, and
positioning or fixing said implant in place at said surgical site to create a fixed implant,
wherein, said expanded implant has a fixed unitary shape which is larger than that which could have passed through said portal.

11. The method of surgery of claim 10, wherein said allograft unitary implant is a molded tissue.

12. The method of surgery of claim 11, wherein said molded tissue comprises demineralized bone matrix (DBM).

13. The method of surgery of claim 12, wherein said molded tissue further comprises a component derived from a human tissue slurry.

14. The method of surgery of claim 10, wherein said allograft unitary implant is rolled in the compressed dehydrated form.

15. The method of surgery of claim 10, wherein said expanded implant is a square, rectangle, circle, triangle or oval.

16. A biological graft, consisting essentially of human tissue components comprising;
a bone block of cortical bone, cancellous bone or both, said bone block having at least one porous or semi-porous surface; and
a molded tissue component comprising demineralized bone matrix (DBM) and a human tissue slurry;
wherein said molded tissue component is integrated into said porous or semi-porous surface of said bone block.

17. The graft of claim 16, wherein said molded tissue component is frozen and lyophilized along at least a portion of at least one outer surface of said bone block;

18. The graft of claim 16, wherein said bone block further comprises at least one slot, groove or hole to increase penetration of said molded tissue component.

19. The graft of claim 16, wherein said molded tissue component covers at least two sides of said bone block and substantially fills a passageway between at least two of said at least two sides.

20. The graft of claim 16, wherein said bone block is in the shape of a wedge, trapezoid, plank or ring.

21. A dehydrated, packaged and sterilized molded human tissue graft in the form of a compressed sheet having an initial dehydrated thickness and an initial dehydrated compressed shape
wherein said graft in said initial dehydrated compressed shape is small enough to be passed into a surgical site within a human patient through a minimally invasive access portal having a cross sectional area, and
wherein said graft in said initial dehydrated compressed shape is rehydratable to return to a hydrated thickness greater than that of said initial dehydrated thickness at said surgical site, and to a hydrated size larger than said cross-sectional area.
